(19) United States
(12) Patent Application Publication
(10) Pub. No.: US 2026/0298170 A1
(43) Pub. Date: Oct. 1, 2026

(54) APPARATUSES, PROCESSES, AND SYSTEMS INCLUDING FUEL COMPOSITION DETERMINATION

(71) Applicant: CLUMMINS INC., Columbus, IN (US)

(72) Inventors: Jonathan A. Groeper, Bargersville, IN (US); Jennifer Kay Light-Holets, Greenwood, IN (US); Corey W. Trobaugh, Columbus, IN (US); Joshua Warriner, Hope, IN (US)

(21) Appl. No.: 19/476,778

(22) PCT Filed: Apr. 23, 2024
(86) PCT No.: PCT/US2024/025755

§ 371 Date: Oct. 21, 2025

Related U.S. Application Data
(60) Provisional application No. 63/498,393, filed on Apr. 26, 2023.

(51) Int. Cl. F02D 41/22; G01N 33/28

(52) U.S. Cl. CPC F02D 41/22; G01N 33/2835; F02D 2041/224; F02D 2200/0606; F02D 2200/0611

(57) ABSTRACT

A powertrain system includes a fuel sensor system adapted to provide an output indicative of a plurality of characteristics of fuel in a fuel supply system adapted to supply fuel to an engine, the plurality of characteristics including information indicative of a fuel temperature and one or more of a fuel density, a fuel viscosity, and a fuel dielectric. An electronic control system in operative communication with the sensor system and configured to receive the output of the fuel sensor system, process the output to determine at a fuel composition parameter, and utilize the fuel composition parameter in controlling one or more operations of the powertrain system.

### CROSS-REFERENCE TO RELATED APPLICATIONS

The present application claims the benefit of and priority to U.S. Application No. 63/498,393 filed Apr. 26, 2023, and the same here hereby incorporated by reference.

### TECHNICAL FIELD

The present application relates to apparatuses, methods, systems, and techniques for determining fuel composition and more particularly, but not exclusively, to apparatuses, methods, systems, and techniques for determining variation in fuel composition and adapting operation of an engine system in response to the variation.

### BACKGROUND

The number and variety of fuel compositions useful for internal combustions continues to evolve and expand and may include blended fuels such as blends of conventional diesel and biodiesel and blends of gasoline and ethanol. The combustion performance of blended fuels can vary significantly with the relative amount or percentage of constituent fuels (e.g., percentage biodiesel or percentage ethanol). This poses challenges for engine controls which would benefit from knowledge of the fuel composition being provided for combustion. These challenges are further complicated by the fact that blended fuel compositions may vary over different refueling events, due to degradation of fuel constituents, or fuel contamination (e.g., the presence of water or lube oil). A number of proposals have been made for evaluating fuel composition including, for example, infrared spectroscopy, nuclear magnetic resonance spectroscopy, and gas chromatography. Existing proposals suffer from a number of disadvantages and shortcomings including those respecting complexity, cost, reliability, robustness, and others. There remains a significant need for the unique apparatuses, processes, systems, and techniques of the present disclosure.

### DISCLOSURE OF EXAMPLE EMBODIMENTS

For the purposes of clearly, concisely, and exactly describing example embodiments of the present disclosure, the manner, and process of making and using the same, and to enable the practice, making and use of the same, reference will now be made to certain example embodiments, including those illustrated in the figures, and specific language will be used to describe the same. It shall nevertheless be understood that no limitation of the scope of the invention is thereby created, and that the invention includes and protects such alterations, modifications, and further applications of the example embodiments as would occur to one skilled in the art.

### SUMMARY OF THE DISCLOSURE

Example embodiments include unique apparatuses, methods, systems, and techniques for determining fuel composition. Further embodiments, forms, objects, features, advantages, aspects, and benefits shall become apparent from the following description and drawings.

### BRIEF DESCRIPTION OF THE DRAWINGS

FIG. 1 is a schematic diagram illustrating certain aspects of an example engine system including an example fueling system.

FIG. 2 is a schematic diagram illustrating certain aspects of example controls.

FIG. 3 is a flow diagram illustrating certain aspects of an example process.

FIG. 4 is a flow diagram illustrating certain aspects of an example process.

FIG. 5 is a flow diagram illustrating certain aspects of an example process.

FIG. 6 is a graph illustrating an example empirically determined data set which may be utilized by the controls and processes of the present disclosure.

FIG. 7 is a graph illustrating an example empirically determined data set which may be utilized by the controls and processes of the present disclosure.

FIG. 8 is a graph illustrating an example empirically determined data set which may be utilized by the controls and processes of the present disclosure.

### DETAILED DESCRIPTION OF EXAMPLE EMBODIMENTS

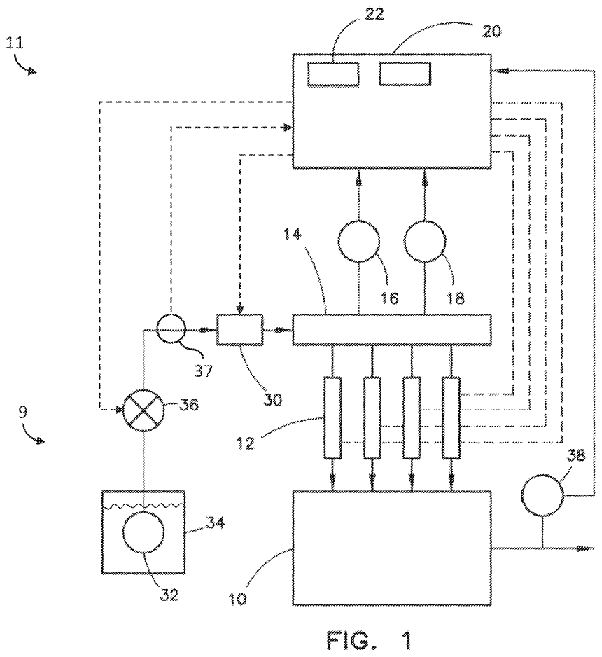

With reference to FIG. 1, there is illustrated a powertrain system 11 (also referred to herein as system 11) comprising an engine 10 including a fueling system 9. The engine 10 may be an internal combustion engine, including but not limited to a compression-ignition engine, using diesel or other suitable fuel, or a spark-ignition engine using gasoline or other suitable fuels. Engine 10 may have one or more combustion cylinders (not depicted) to generate mechanical power from the combustion of a fuel. The fuel injectors 12 are in fluid communication with respective combustion cylinders of the engine 10 and are structured to introduce the fuel into respective combustion cylinders. Though four fuel injectors 12 are depicted in FIG. 1, engine 10 may include fewer or greater numbers of fuel injectors 12. In certain embodiments, engine 10 may include one fuel injector 12 for each cylinder.

In shall be appreciated that system 11 may be provided in a number of forms including, for example as a terrestrial vehicle powertrain system (e.g., an on-highway vehicle powertrain system or off-highway vehicle powertrain system), a work machine powertrain system, a genset powertrain system, or a hydraulic fracturing rig powertrain system, to name several non-limiting examples. In shall also be appreciated that system 11 may include a number of other components which are not illustrated in FIG. 1 including, for example, a valve control system, a turbocharger system, and exhaust gas recirculation system, and a transmission, non-engine prime mover components such as energy storage systems and electric motors, to name several non-limiting examples.

In the illustrated embodiment, the fueling system 9 is configured and provided as a high-pressure common-rail fuel injection system including a plurality of fuel injectors 12 in fluid communication with a common fuel rail 14, which supplies fuel at relatively high pressure to each fuel injector 12. Fuel may be supplied to the common fuel rail 14 by a high-pressure pump 30. In certain embodiments, the high-pressure pump may be fed by a relatively low-pressure fuel circuit including a booster pump 32, which may be immersed in a tank 34 containing the fuel. A fuel regulator 36 may control the flow of fuel from tank 34 to the high-pressure pump 30.

System 11 further includes an electronic control system (ECS) 20 in communication with engine 10 and configured to control one or more aspects of engine 10, including controlling the injection of fuel into engine 10 via the fuel injectors 12. Accordingly, ECS 20 may be in communication with the fuel injectors 12 and configured to command each fuel injector 12 on and off at prescribed times to inject fuel into the engine 10 as desired. ECS 20 include at least one electronic control unit (ECU) 22 configured to execute operations of ECS 20 as described further herein and, in some embodiment, may include additional ECUs configured to execute operations of ECS 20 as described further herein.

It shall be appreciated, that that composition or constituents of fuel provided to engine 10 by fuel system 9 may vary over time, for example, due to refueling events with different fuels, fuel degradation, or introduction or presence of contaminants such as water or engine lubrication oil into the fuel being provided. A sensor system 37 comprising one or more sensors may be configured to sense one or more parameters of fuel being supplied to engine 12 by fuel system 9. In some embodiments the fuel sensor system 37 may be provided as a single sensor in operative communication ECS 20, and configured to sense a temperature, a density, a viscosity, and a dielectric and provide the same as a multiplexed or encoded signal. In some such embodiments, the multiplexed or encoded signal may comprise a fixed length message encoding values for the temperature, density, viscosity, and dielectric. In some embodiments the fuel sensor system may be provided as a plurality of sensors in operative communication with an electronic control system which are collectively configured to sense a temperature, density, viscosity, and dielectric and provide the same to the electronic control system either as individual signals or as post-sensor multiplexed or encoded signals. While sensor system 37 is illustrated in FIG. 1 at an example position in fuel system 9, it shall be appreciated that sensor system 37, or one or more of its constituent sensors may be provided at a various other points in fuel system 9 and may, in principle be provided at any point or location in fuel system 9 at or between tank 34 and injectors 12 to sense one or more parameters of fuel being supplied to engine 12 by fuel system 9.

ECS 20 may be further structured to control other parameters of engine 10, which may include aspects of engine 10 that may be controlled with an actuator activated by ECS 20. For example, ECS 20 may be in communication with actuators and sensors for receiving and processing sensor input and transmitting actuator output signals. Actuators may include, but not be limited to, fuel injectors 12. The sensors may include any suitable devices to monitor operating parameters and functions of the system 11. For example, the sensors may include a pressure sensor 16 and a temperature sensor 18. The pressure sensor 16 is in communication with the common fuel rail 14 and structured to communicate a measurement of the pressure within the common fuel rail 14 to the ECS 20. The temperature sensor 18 is in communication with the common fuel rail 14 and structured to communicate a measurement of the temperature within the common fuel rail 14 to the ECS 20. In at least one embodiment, system 11 may include an oxygen sensor 38 (e.g., a lambda sensor) in communication with the ECS 20 and structured to determine characteristics of exhaust gases generated and expelled by the engine 10. In one example, oxygen sensor 38 may determine the concentration of oxygen in the exhaust gases as a proxy for the concentration of regulated emissions.

As will be appreciated by the description that follows, the techniques described herein relating to fuel injector or fuel injection parameters can be implemented in ECS 20, which may include one or more controllers for controlling different aspects of the system 11. In one form the ECS 20 comprises one or more electronic control units (ECU) such as an engine control unit or engine control module. The ECS 20 may be comprised of digital circuitry, analog circuitry, or a hybrid combination of both of these types. Also, the ECS 20 may be programmable, an integrated state machine, or a hybrid combination thereof. The ECS 20 may include one or more Arithmetic Logic Units (ALUs), Central Processing Units (CPUs), memories, limiters, conditioners, filters, format converters, or the like which are not shown to preserve clarity. In one form, the ECS 20 is of a programmable variety that executes algorithms and processes data in accordance with operating logic that is defined by programming instructions (such as software or firmware). Alternatively or additionally, operating logic for the ECS 20 may be at least partially defined by hardwired logic or other hardware.

In addition to the types of sensors described herein, any other suitable sensors and their associated parameters may be encompassed by the system and methods. Accordingly, the sensors may include any suitable device used to sense any relevant physical parameters including electrical, mechanical, and chemical parameters of the powertrain system 11. As used herein, the term sensors may include any suitable hardware and/or software used to sense or estimate any engine system parameter and/or various combinations of such parameters either directly or indirectly.

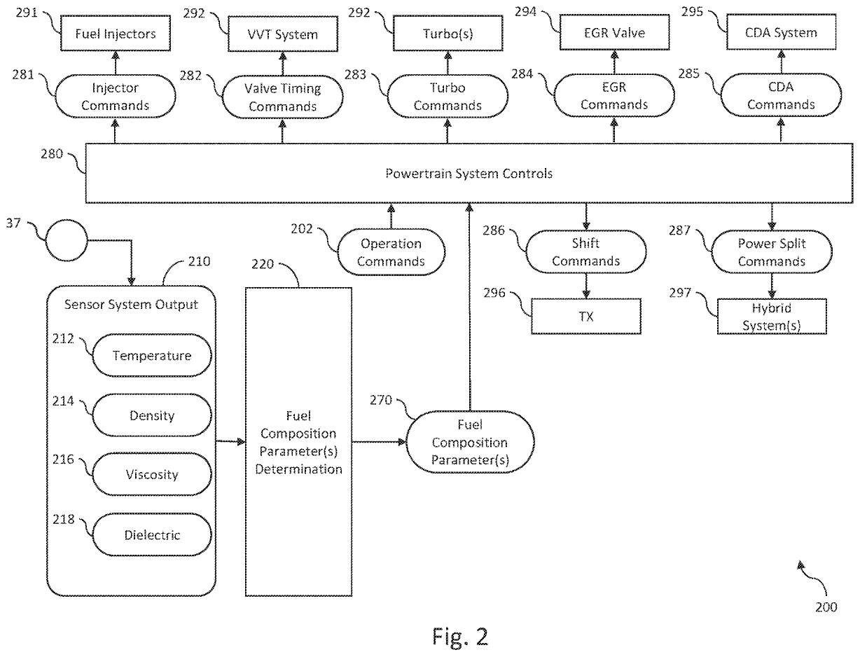

With reference to FIG. 2, there are illustrated example controls 200 which may be implemented in and operated by one or more components of an electronic control system such as ECS 20 or another electronic control system configured for operative communication with a fueling system. In some forms, at least a portion of controls 200 may be implemented in one or mode electronic control units of an electronic control system such as ECU 22 or additional or alternative electronic control units.

Controls 200 are configured to receive sensor system output 210 from sensor system 37. In the illustrated example, sensor system output 210 may include one or more of temperature information 212, density information 214, viscosity information 216, and dielectric information 218 of the fuel being analyzed by sensor system 37. Sensor system output 210 is provided to fuel composition parameter(s) determination block 220 (also referred to herein as block 220). Block 220 may be configured to perform and may execute one or more operations to determine one or more fuel composition parameters. In some embodiments, block 220 may be configured to perform all of or part of one or more of the operations described in connection with process 300, process 400, or process 500 which are further described herein. Block 220 determines and outputs fuel composition parameter(s) 270.

Fuel composition parameter(s) 270 are provided to one or more powertrain system controls 280 which are also provided with one or more operation commands 202 such as accelerator pedal commands, cruise control commands, autonomous driving system commands, o other powertrain operation commands. The one or more powertrain system controls 280 are configured to utilize the fuel composition parameter(s) 270 in controlling one or more operations of the powertrain system such as system 11.

The one or more powertrain system controls 280 may be implemented and provided as one more components of an electronic control system such as one or more electronic control units (e.g., ECU 22 and/or other electronic control units) and/or by other electronic control system components. Powertrain system controls 280 are configured to receive the fuel composition parameter(s) 270, and utilize the fuel composition parameter(s) 270 in controlling one or more operations of a powertrain system such as system 11. Non-limiting examples of the powertrain system that may be controlled using the fuel composition parameter(s) 270 include controlling or adjusting one or more of fueling quantity, fuel injection timing, number of fueling pulses, cylinder intake or cylinder exhaust valve(s) opening or closing, VGT, wastegate, EGR commands, transmission shift schedule, cylinder de-activation (including skip-fire), or electric motor powersplit (e.g., in the case of a hybrid powertrain system). More specifically, in the illustrated example powertrain system controls 280 may utilize the fuel composition parameter(s) 270 to determine injector commands 281 which are adapted to control operation of one or more fuel injectors 291, valve timing commands 282 which are adapted to control operation of the variable valve timing (VVT) system 292, turbocharger commands 283 which are adapted to control operation of one or more turbochargers 293, exhaust gas recirculation (EGR) commands 284 which are adapted to control operation of an EGR valve to 294, cylinder deactivation (CDA) commands 285 which are configured to control operation of a CDA system 295, shift commands 286 which are adapted to control shifting operation of transmission 296, and/or power split commands 287 which are adapted to control operation of one or more hybrid systems 297.

Controls 200 is one example of controls adapted to utilize a sensor system including one or more sensors capable of measuring viscosity, dielectric, density and temperature to determine biodiesel content on a working engine. Such systems offer the capability for an engine to adjust itself for whatever fuel an operator uses.

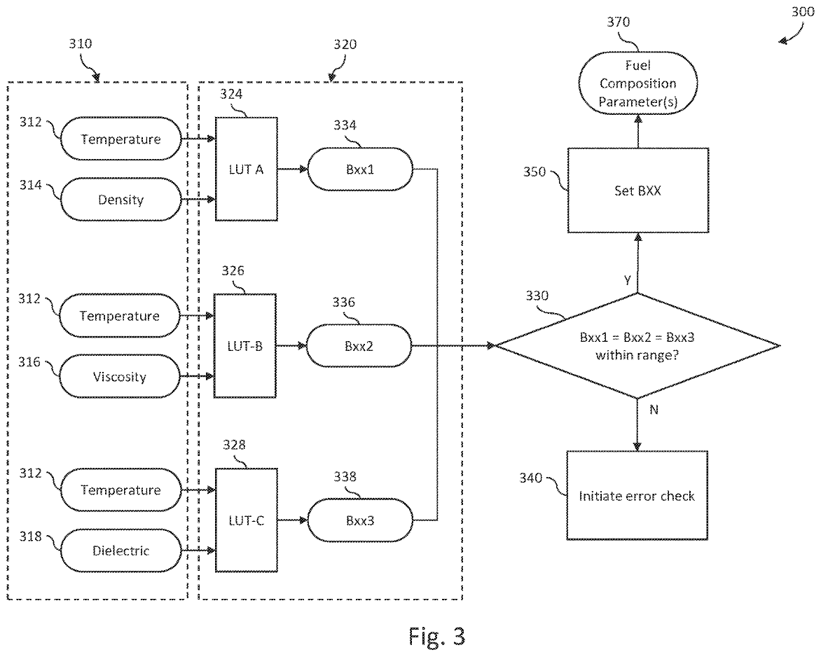

With reference to FIG. 3, there is illustrated an example process 300 for operating an electronic control system (e.g., ECS 20 or another electronic control system), in operative communication with a fueling system (e.g., fueling system 9 or another fueling system). Process 300 may be implemented in and performed by one or more components of an electronic control system such as one or more electronic control units (e.g., ECU 22 and/or other electronic control units) and/or by other electronic control system components.

After being initiated, process 300 performs one or more operations 310 at which one or more outputs of a fuel sensor system (e.g., sensor system 37 or another fuel system) is received by an electronic control system (e.g., ECS 20 or another electronic control system). In the illustrated example, the one or more outputs of the fuel sensor system include temperature parameter 312, density parameter 314, viscosity parameter 316, and dielectric parameter 318. Temperature parameter 312 may be provided in a number of forms, for example, as one or more values explicitly indicative of a temperature of fuel contacting or otherwise associated with the fuel sensor system, or may be provided implicitly such as by providing one or more of density parameter 314, viscosity parameter 316, and dielectric parameter 318 as values respectively indicating density, viscosity, and dielectric parameters as a function of temperature. Density parameter 314 may be provided in a number of forms, for example, as one or more values indicative of a density of fuel contacting or otherwise associated with the fuel sensor system, such as density, specific volume, or other expressions of an intrinsic ratio of or relationship between mass and volume of the fuel. Viscosity parameter 316 may be provided in a number of forms, for example, or more values indicative of a viscosity of fuel contacting or otherwise associated with the fuel sensor system such as dynamic viscosity, kinematic viscosity, or other parameters indicative of resistance to deformation. Dielectric parameter 318 may be provided in a number of forms, for example, as one or more values indicative of a dielectric constant of fuel contacting or otherwise associated with the fuel sensor system or as one or more values correlated with a dielectric constant, such as absolute permittivity, relative permittivity, or other correlative values.

It shall be appreciated that the one or more operations 310 may be performed as a single operation or multiple operations. For example, in some embodiments the fuel sensor system may be provided as a single sensor in operative communication with an electronic control system, the single sensor being configured to sense temperature parameter 312, density parameter 314, viscosity parameter 316, and dielectric parameter 318 and provide the same as a multiplexed or encoded signal. In some such embodiments, the multiplexed or encoded signal may comprise a fixed length message encoding values for temperature parameter 312, density parameter 314, viscosity parameter 316, and dielectric parameter 318. In some embodiments the fuel sensor system may be provided as a plurality of sensors in operative communication with an electronic control system which are collectively configured to sense temperature parameter 312, density parameter 314, viscosity parameter 316, and dielectric parameter 318 and provide the same to the electronic control system either as individual signals or as post-sensor multiplexed or encoded signals.

From the one or more operation 310, process 300 proceeds to one or more operations operation 320 which process the values received via the one or more operations 310. In the illustrated example the one or more operation 320 include operation 324, operation 326, and operation 328. It shall be appreciated that operation 324, operation 326, and operation 328 may be performed as parallel or concurrent operations, as series or sequential operations, as distinct or separate operations, or is combined or unitary operations wherein the functionality of two or more of operation 324, operation 326, and operation 328 is combined in a single lookup operation.

At operation 324 a lookup operator (e.g., a lookup table, map, or other data structure) receives temperature parameter 312 and density parameter 314 and in response to the received inputs outputs a fuel composition parameter 334. In the illustrated example, the fuel composition parameter 334, comprises a biodiesel content value Bxx1 which indicates a percentage, fraction, or other quantitative metric of biodiesel present in the fuel being analyzed. Operation 324 may be implemented via one or more lookup tables comprising empirically established relationships between temperature and density for a plurality of biodiesel percentage values.

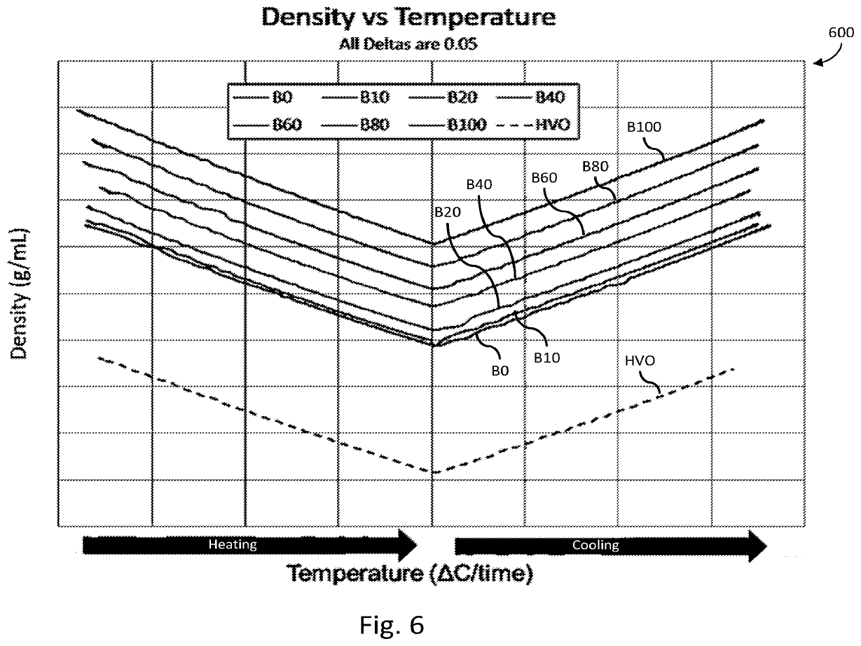

Graph 600 of FIG. 6 illustrates an example empirical data set which may be utilized to establish the parameters utilized by operation 324. Graph 600 depicts curves B100, B80, B60, B40, B20, B10, and B0 of fuel density as a function of temperature for both a heating cycle and a cooling cycle for biodiesel blends of 100%, 80%, 60%, 40%, 20%, 10%, and 0% biodiesel, respectively. Graph 600 also depicts curve HVO of fuel density as a function of temperature for both a heating cycle and a cooling cycle for hydrotreated vegetable oil (HVO). Curve HVO further illustrates an impact of aromatic content relative to curve BO as HVO has zero aromatic content and the aromatic content BO may be non-zero and may vary with source region. These relationships or other empirically derived relationships may be encoded into the logic utilized by operation 324. Other embodiments also contemplate that techniques other than lookup tables may be utilized. For example some embodiments may utilize multi-order polynomial equations, or other techniques calculations or computational techniques rather than lookup tables.

At operation 326 a lookup operator (e.g., a lookup table, map, or other data structure) receives temperature parameter 312 and viscosity parameter 316 and in response to the received inputs outputs a fuel composition parameter 336. In the illustrated example, the fuel composition parameter 336, comprises a biodiesel content value Bxx2 which indicates a percentage, fraction, or other quantitative metric of biodiesel present in the fuel being analyzed. Operation 326 may be implemented via one or more lookup tables comprising empirically established relationships between temperature and density for a plurality of biodiesel percentage values.

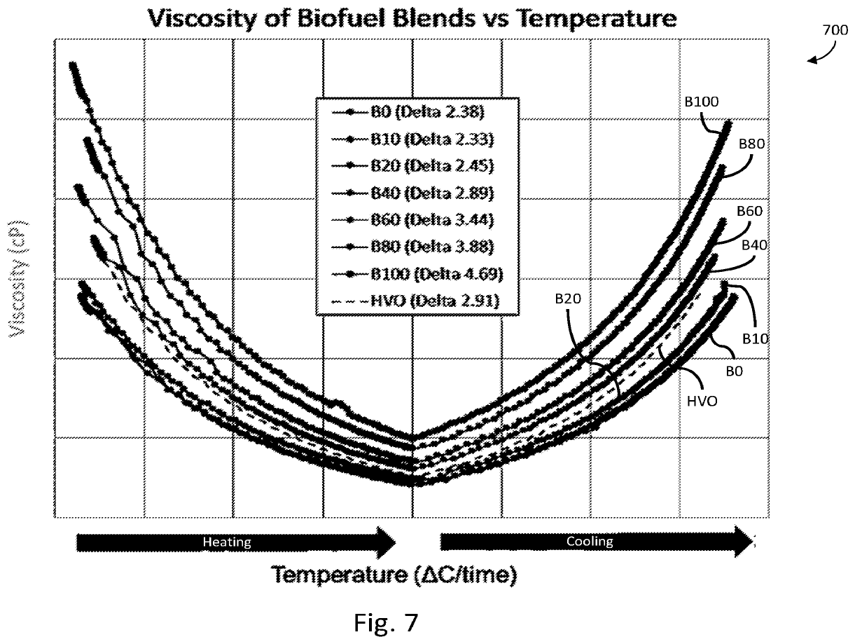

Graph 700 of FIG. 7 illustrates an example empirical data set which may be utilized to establish the parameters utilized by operation 324. Graph 700 depicts curves B100, B80, B60, B40, B20, B10, and BO of fuel viscosity as a function of temperature for both a heating cycle and a cooling cycle for biodiesel blends of 100%, 80%, 60%, 40%, 20%, 10%, and 0% biodiesel, respectively. Graph 700 also depicts curve HVO of fuel density as a function of temperature for both a heating cycle and a cooling cycle for hydrotreated vegetable oil (HVO). Curve HVO further illustrates an impact of aromatic content relative to curve BO as HVO has zero aromatic content and the aromatic content BO may be non-zero and may vary with source region. These relationships or other empirically derived relationships may be encoded into the logic utilized by operation 326. Other embodiments also contemplate that techniques other than lookup tables may be utilized, for example, the techniques described in connection with lookup operation.

At operation 328 a lookup operator (e.g., a lookup table, map, or other data structure) receives temperature parameter 312 and dielectric parameter 318 and in response to the received inputs outputs a fuel composition parameter 338. In the illustrated example, the fuel composition parameter 338, comprises a biodiesel content value Bxx3 which indicates a percentage, fraction, or other quantitative metric of biodiesel present in the fuel being analyzed. Operation 328 may be implemented via one or more lookup tables comprising empirically established relationships between temperature and density for a plurality of biodiesel percentage values.

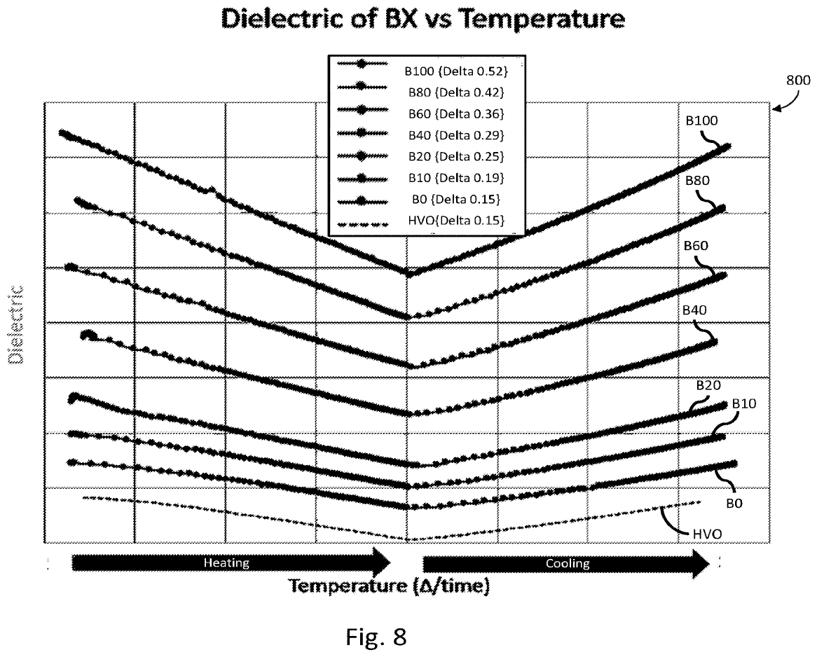

Graph 800 of FIG. 8 illustrates an example empirical data set which may be utilized to establish the parameters utilized by operation 324. Graph 800 depicts curves B100, B80, B60, B40, B20, B10, and BO of fuel dielectric as a function of temperature for both a heating cycle and a cooling cycle for biodiesel blends of 100%, 80%, 60%, 40%, 20%, 10%, and 0% biodiesel, respectively. Graph 800 also depicts curve HVO of fuel density as a function of temperature for both a heating cycle and a cooling cycle for hydrotreated vegetable oil (HVO). Curve HVO further illustrates an impact of aromatic content relative to curve BO as HVO has zero aromatic content and the aromatic content BO may be non-zero and may vary with source region. These relationships or other empirically derived relationships may be encoded into the logic utilized by operation 328. Other embodiments also contemplate that techniques other than lookup tables may be utilized, for example, the techniques described in connection with lookup operation.

It shall be appreciated that the empirical data sets depicted by graph 600, graph 700, and/or graph 800 may be extended and augmented in a number of manners. In some embodiments, one or more such data sets may be extended or augmented to account for the presence of water in the fuel being analyzed. In such embodiments a plurality of data sets which have varying amounts of water present in the fuel being analyzed or otherwise analogous to the data set depicted by graph 600, graph 700, and/or graph 800 may be utilized in the resulting associated operations performed using the data set may better account for variation in the amount of water present in the fuel being analyzed.

In some embodiments, one or more such data sets may be extended or augmented to account for the presence of an engine lubrication oil in the fuel being analyzed. In such embodiments a plurality of data sets which have varying amounts of engine lubrication oil present in the fuel being analyzed or otherwise analogous to the data set depicted by graph 600, graph 700, and/or graph 800 may be utilized in the resulting associated operations performed using the data set may better account for variation in the amount of lubrication oil present in the fuel being analyzed.

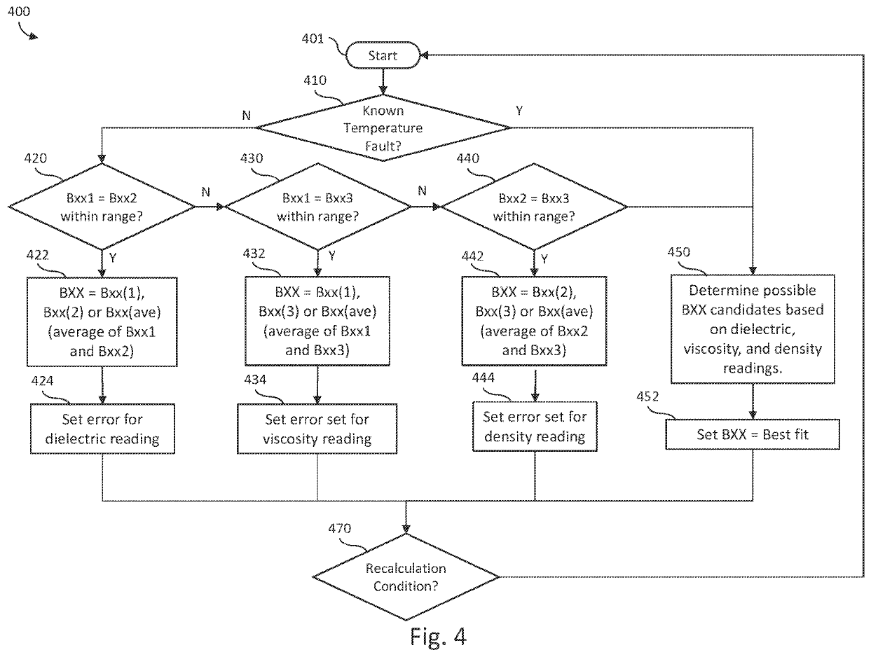

Fuel composition parameter 334, fuel composition parameter 336, fuel composition parameter 338 are provided to conditional 330. Conditional 330 evaluates whether biodiesel content value Bxx1, biodiesel content value Bxx2, and biodiesel content value Bxx3 are equal to one another within a predetermined range or variance. If conditional 330 evaluates negative, process 300 proceeds to operation 340 which initiates one or more error check processes, for example, process 400 illustrated in FIG. 4 and further described herein. If conditional 330 evaluates affirmative, process 300 proceeds to operation 350 which sets a biodiesel content value BXX using one or more of biodiesel content value Bxx1, biodiesel content value Bxx2, and biodiesel content value Bxx3 and outputs one or more fuel composition parameters 370 comprising the biodiesel content value BXX.

With reference to FIG. 4, there is illustrated an example process 400 for operating an electronic control system (e.g., ECS 20 or another electronic control system), in operative communication with a fueling system (e.g., fueling system 9 or another fueling system). Process 400 may be implemented in and performed by one or more components of an electronic control system such as one or more electronic control units (e.g., ECU 22 and/or other electronic control units) and/or by other electronic control system components.

Process 400 is initiated at start operation 401, for example, if called or initiated by operation 340. From start operation 401, process 400 proceeds to conditional 410 which evaluates whether a known temperature fault condition is true. If conditional 410 evaluates affirmative, process 400 proceeds to conditional 450 whose operation is further described herein.

If conditional 410 evaluates negative, process 400 proceeds to conditional 420 which evaluates whether biodiesel content value Bxx1 is equal to biodiesel content value Bxx2. If conditional 420 evaluates affirmative, process 400 proceeds to operation 422 which sets biodiesel content value BXX using at least one of biodiesel content Bxx1 and biodiesel content value Bxx2, for example, by setting biodiesel content BXX equal to biodiesel content Bxx1, by setting biodiesel content BXX equal to biodiesel content Bxx2, or by setting biodiesel content BXX equal to an average of biodiesel content Bxx1 and biodiesel content Bxx2 or another metric calculated using biodiesel content Bxx1 and biodiesel content Bxx2.

From operation 422, process 400 proceeds operation 424 which sets an error condition for a dielectric sensor reading. From operation 424, process 400 proceeds to conditional 470 which is further described herein.

If conditional 420 evaluates negative, process 400 proceeds to conditional 430 which evaluates whether biodiesel content value Bxx1 is equal to biodiesel content value Bxx3. If conditional 430 evaluates affirmative, process 400 proceeds to operation 432 which sets biodiesel content value BXX using at least one of biodiesel content Bxx1 and biodiesel content value Bxx3,for example, by setting biodiesel content BXX equal to biodiesel content Bxx1, by setting biodiesel content BXX equal to biodiesel content Bxx3, or by setting biodiesel content BXX equal to an average of biodiesel content Bxx1 and biodiesel content Bxx3 or another metric calculated using biodiesel content Bxx1 and biodiesel content Bxx3.

From operation 432, process 400 proceeds operation 434 which sets an error condition for a viscosity sensor reading. From operation 434, process 400 proceeds to conditional 470 which is further described herein.

If conditional 430 evaluates negative, process 400 proceeds to conditional 440 which evaluates whether biodiesel content value Bxx2 is equal to biodiesel content value Bxx3. If conditional 440 evaluates affirmative, process 400 proceeds to operation 442 which sets biodiesel content value BXX using at least one of biodiesel content Bxx2 and biodiesel content value Bxx3, for example, by setting biodiesel content BXX equal to biodiesel content Bxx2, by setting biodiesel content BXX equal to biodiesel content Bxx3, or by setting biodiesel content BXX equal to an average of biodiesel content Bxx2 and biodiesel content Bxx3 or another metric calculated using biodiesel content Bxx2 and biodiesel content Bxx3.

From operation 442, process 400 proceeds operation 434 which sets an error condition for a viscosity sensor reading. From operation 434, process 400 proceeds to conditional 470 which is further described herein.

If conditional 440 evaluates negative, process 400 proceeds to operation 450. Likewise, as noted above, if conditional 410 evaluates affirmative, process 400 proceeds to operation 450. Operation 450 determines possible candidate values for setting biodiesel content BXX based on the available biodiesel content value Bxx1, biodiesel content value Bxx2, and biodiesel content value Bxx3, and their associated dielectric, viscosity, and density information. Operation 450 may use a number of techniques to determine possible candidate values for setting biodiesel content BXX. For example operation 450 may discard one or more outlier values or greatest outlier value from among the available biodiesel content value Bxx1, biodiesel content value Bxx2, and biodiesel content value Bxx3.

From operation 450, process 400 proceeds to operation 452 which sets a value of biodiesel content BXX in response to the possible candidate values identified and operation 450. Operation 452 may, for example, identify a candidate value which deviates least from an expected value, calculate an average of multiple candidate values, calculate a weighted average of multiple candidate values weighted according to their parent deviation from expected value, or utilize a variety of other statistical an error reduction techniques as will occur to one skilled in the art with the benefit and insight of the present disclosure. It shall likewise be appreciated by one of skill in the art that if one of these is not available (e.g., is not provided by a sensor or is provided with an error indication that it is unsuitable for use), process 400 can proceed with the available values From operation 452, and as noted above from operation 424, operation 434, and operation 444, process 400 proceeds to conditional 470. Conditional 470 evaluates whether one or more recalculation conditions is true. The one or more recalculation conditions may include one or more of a key-on condition, a change in another temperature value (e.g., an oil or coolant temperature value) or an excursion of such temperature value relative to a threshold, an engine fault, and aftertreatment fault, an engine or aftertreatment NOx or reductant consumption greater than expected range for known operating condition, a refueling event or other recalculation condition triggers as will occur to one of skill in the art with the benefit and insight of the present disclosure. If conditional 470 evaluates negative, process 400 repeats conditional 470. If conditional 470 evaluates affirmative, process 400 proceeds to start operation 401 and from there proceeds as described above. Alternatively, process 400 may and subsequently be reinitiated, recalled or otherwise repeated.

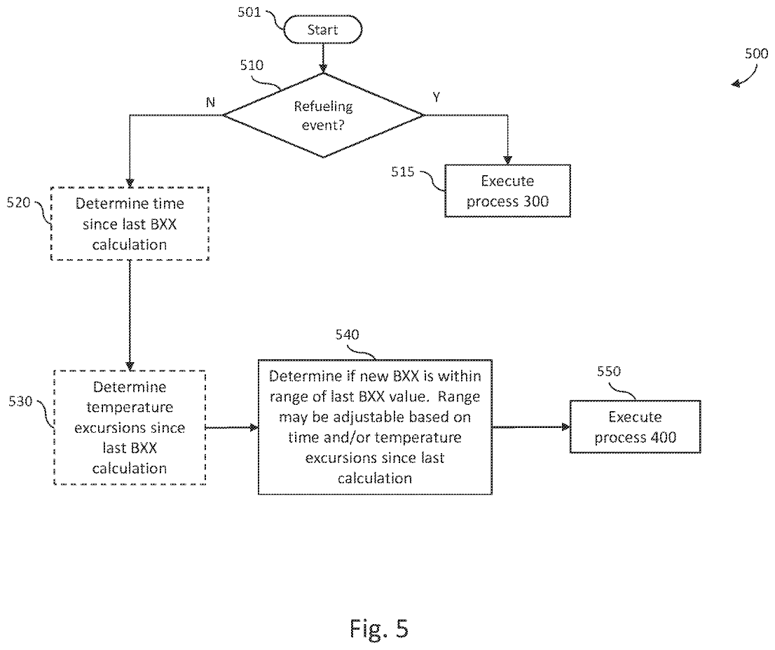

With reference to FIG. 5 there is illustrated an example process 500for operating an electronic control system (e.g., ECS 20 or another electronic control system), in operative communication with a fueling system (e.g., fueling system 9 or another fueling system). Process 500 may be implemented in and performed by one or more components of an electronic control system such as one or more electronic control units (e.g., ECU 22 and/or other electronic control units) and/or by other electronic control system components.

Process 500 is initiated at start operation 501 and proceeds to conditional 510 which evaluates whether a refueling event condition is true. If conditional 510 evaluates affirmative, process 500 proceeds to operation 515 which may execute or call process 300 or another process configured to determine a biodiesel content value. If conditional 510 evaluates negative, process 500 proceeds to operation 520 determines a time since a last biodiesel content determination.

From operation 520, process 500 proceeds to operation 530. Alternatively, in some embodiments, operation 520 may be omitted and process 500 may proceed from conditional 510 to operation 530. Operation 530 determines a number of temperature excursions since a last biodiesel content determination.

From operation 530, process 500 proceeds to operation 540. Alternatively, in some embodiments, operation 530 may be omitted and process 500 may proceed from conditional 510 or operation 520 (if present) to operation 540. Operation 540 determines if new biodiesel content value is within a range of prior biodiesel content value. The range may be adjustable (e.g., increased or decreased) based on time and/or temperature excursions since a prior biodiesel content determination. From operation 540, process 500 proceeds to operation 550 which executes process 400 or another error checking process.

As shown by this detailed description, the present disclosure contemplates multiple and various embodiments, including, without limitation, the following example embodiments. A first example embodiment is a powertrain system comprising: a fuel sensor system adapted to provide an output indicative of a plurality of characteristics of fuel in a fuel supply system adapted to supply fuel to an engine, the plurality of characteristics including information indicative of a fuel temperature and one or more of a fuel density, a fuel viscosity, and a fuel dielectric; an electronic control system in operative communication with the fuel sensor system and configured to: receive the output of the fuel sensor system, process the output to determine at a fuel composition parameter, and utilize the fuel composition parameter in controlling one or more operations of the powertrain system.

A second example embodiment includes the features of the first example embodiment, wherein the plurality of characteristics include information indicative at least two of the fuel density, the fuel viscosity, and the fuel dielectric, and the electronic control system is configured to check for an error condition by evaluating at least said at least two of the fuel density, the fuel viscosity, and the fuel dielectric.

A third example embodiment includes the features of the second example embodiment, wherein the electronic control system is configured to determine a first biodiesel content value using the fuel temperature and a first one of the at least two of the fuel density, the fuel viscosity, and the fuel dielectric, and to determine a second biodiesel content value using the fuel temperature and a second one of the at least two of the fuel density, the fuel viscosity, and the fuel dielectric.

A fourth example embodiment includes the features of the third example embodiment, wherein the electronic control system is configured to evaluate the first biodiesel content value and the second biodiesel content value to check for the error condition.

A fifth example embodiment includes the features of the third example embodiment, wherein the electronic control system is configured to utilize the first biodiesel content value and the second biodiesel content value to determine the fuel composition parameter.

A sixth example embodiment includes the features of the third example embodiment, wherein the electronic control system is configured to set the fuel composition parameter as one of the first biodiesel content value, the second biodiesel content value, and an average of the first biodiesel content value, and the second biodiesel content value.

A seventh example embodiment includes the features of the first example embodiment, wherein the plurality of characteristics include information indicative all of the fuel density, the fuel viscosity, and the fuel dielectric, and the electronic control system is configured to check for an error condition by comparing all of the fuel density, the fuel viscosity, and the fuel dielectric.

Am eighth example embodiment includes the features of the seventh example embodiment, wherein the electronic control system is configured to determine a first biodiesel content value using the fuel temperature and the fuel density, determine a second biodiesel content value using the fuel temperature and the fuel viscosity, and determine a third biodiesel content value using the fuel temperature and the fuel dielectric.

A ninth example embodiment includes the features of the eighth example embodiment, wherein the electronic control system is configured to utilize the first biodiesel content value, the second biodiesel content value, and the third biodiesel content value to determine to determine the fuel composition parameter.

A tenth example embodiment includes the features of the ninth example embodiment, wherein the electronic control system is configured to set the fuel composition parameter as one of the first biodiesel content value, the second biodiesel content value, the second biodiesel content value, and an average of two or more of the first biodiesel content value, the second biodiesel content value, and the third biodiesel content value.

An eleventh example embodiment is a method of controlling a powertrain system, the method comprising: receiving with an electronic control system an output of a fuel sensor system, the output being indicative of a plurality of characteristics of fuel in a fuel supply system adapted to supply fuel to an engine, the plurality of characteristics including information indicative of a fuel temperature and one or more of a fuel density, a fuel viscosity, and a fuel dielectric, determining a fuel composition parameter by processing the output with the electronic control system, and controlling with the electronic control system one or more operations of the powertrain system, the controlling utilizing the fuel composition parameter.

A twelfth example embodiment includes the features of the eleventh example embodiment, wherein the plurality of characteristics include information indicative at least two of the fuel density, the fuel viscosity, and the fuel dielectric, and the electronic control system is configured to check for an error condition by evaluating at least said at least two of the fuel density, the fuel viscosity, and the fuel dielectric.

A thirteenth example embodiment includes the features of the twelfth example embodiment, comprising: determining with the electronic control system a first biodiesel content value using the fuel temperature and a first one of the at least two of the fuel density, the fuel viscosity, and the fuel dielectric, and determining with the electronic control system a second biodiesel content value using the fuel temperature and a second one of the at least two of the fuel density, the fuel viscosity, and the fuel dielectric.

A fourteenth example embodiment includes the features of the thirteenth example embodiment, comprising evaluating with the electronic control system the first biodiesel content value and the second biodiesel content value to check for the error condition.

A fifteenth example embodiment includes the features of the thirteenth example embodiment, comprising determining the fuel composition parameter using the first biodiesel content value and the second biodiesel content value.

A sixteenth example embodiment includes the features of the thirteenth example embodiment, comprising setting the fuel composition parameter as one of the first biodiesel content value, the second biodiesel content value, and an average of the first biodiesel content value, and the second biodiesel content value.

A seventeenth example embodiment includes the features of the eleventh example embodiment, wherein the plurality of characteristics include information indicative all of the fuel density, the fuel viscosity, and the fuel dielectric, and the electronic control system is configured to check for an error condition by comparing all of the fuel density, the fuel viscosity, and the fuel dielectric.

An eighteenth example embodiment includes the features of the seventeenth example embodiment, wherein comprising determining a first biodiesel content value using the fuel temperature and the fuel density, a second biodiesel content value using the fuel temperature and the fuel viscosity, and a third biodiesel content value using the fuel temperature and the fuel dielectric.

A nineteenth example embodiment includes the features of the eighteenth example embodiment, comprising utilizing the first biodiesel content value, the second biodiesel content value, and the third biodiesel content value to determine to determine the fuel composition parameter.

A twentieth example embodiment includes the features of the nineteenth example embodiment, comprising setting the fuel composition parameter as one of the first biodiesel content value, the second biodiesel content value, the second biodiesel content value, and an average of two or more of the first biodiesel content value, the second biodiesel content value, and the third biodiesel content value.

It shall be appreciated that terms such as “a non-transitory memory,”“ .” “a non-transitory memory medium,” and “a non-transitory memory device” refer to a number of types of devices and storage mediums which may be configured to store information, such as data or instructions, readable or executable by a processor or other components of a computer system and that such terms include and encompass a single or unitary device or medium storing such information, multiple devices or media across or among which respective portions of such information are stored, and multiple devices or media across or among which multiple copies of such information are stored.

It shall be appreciated that terms such as “determine,” “determined,” “determining” and the like when utilized in connection with a control method or process, an electronic control system or controller, electronic controls, or components or operations of the foregoing refer inclusively to a number of acts, configurations, devices, operations, and techniques including, without limitation, calculation or computation of a parameter or value, obtaining a parameter or value from a lookup table or using a lookup operation, receiving parameters or values from a datalink or network communication, receiving an electronic signal (e.g., a voltage, frequency, current, or pulse-width modulation (PWM) signal) indicative of the parameter or value, receiving output of a sensor indicative of the parameter or value, receiving other outputs or inputs indicative of the parameter or value, reading the parameter or value from a memory location on a computer-readable medium, receiving the parameter or value as a run-time parameter, and/or by receiving a parameter or value by which the interpreted parameter can be calculated, and/or by referencing a default value that is interpreted to be the parameter value.

While example embodiments of the disclosure have been illustrated and described in detail in the drawings and foregoing description, the same is to be considered as illustrative and not restrictive in character, it being understood that only certain example embodiments have been shown and described and that all changes and modifications that come within the spirit of the claimed inventions are desired to be protected. It should be understood that while the use of words such as preferable, preferably, preferred or more preferred utilized in the description above indicates that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. A powertrain system comprising:
a fuel sensor system adapted to provide an output indicative of a plurality of characteristics of fuel in a fuel supply system adapted to supply fuel to an engine, the plurality of characteristics including information indicative of a fuel temperature and one or more of a fuel density, a fuel viscosity, and a fuel dielectric;
an electronic control system in operative communication with the fuel sensor system and configured to:
receive the output of the fuel sensor system,
process the output to determine at a fuel composition parameter, and
utilize the fuel composition parameter in controlling one or more operations of the powertrain system.

2. The powertrain system of claim 1, wherein the plurality of characteristics include information indicative at least two of the fuel density, the fuel viscosity, and the fuel dielectric, and the electronic control system is configured to check for an error condition by evaluating at least said at least two of the fuel density, the fuel viscosity, and the fuel dielectric.

3. The powertrain system of claim 2, wherein the electronic control system is configured to determine a first biodiesel content value using the fuel temperature and a first one of the at least two of the fuel density, the fuel viscosity, and the fuel dielectric, and to determine a second biodiesel content value using the fuel temperature and a second one of the at least two of the fuel density, the fuel viscosity, and the fuel dielectric.

4. The powertrain system of claim 3, wherein the electronic control system is configured to evaluate the first biodiesel content value and the second biodiesel content value to check for the error condition.

5. The powertrain system of claim 3, wherein the electronic control system is configured to utilize the first biodiesel content value and the second biodiesel content value to determine the fuel composition parameter.

6. The powertrain system of claim 3, wherein the electronic control system is configured to set the fuel composition parameter as one of the first biodiesel content value, the second biodiesel content value, and an average of the first biodiesel content value, and the second biodiesel content value.

7. The powertrain system of claim 1, wherein the plurality of characteristics include information indicative all of the fuel density, the fuel viscosity, and the fuel dielectric, and the electronic control system is configured to check for an error condition by comparing all of the fuel density, the fuel viscosity, and the fuel dielectric.

8. The powertrain system of claim 7, wherein the electronic control system is configured to determine a first biodiesel content value using the fuel temperature and the fuel density, determine a second biodiesel content value using the fuel temperature and the fuel viscosity, and determine a third biodiesel content value using the fuel temperature and the fuel dielectric.

9. The powertrain system of claim 8, wherein the electronic control system is configured to utilize the first biodiesel content value, the second biodiesel content value, and the third biodiesel content value to determine to determine the fuel composition parameter.

10. The powertrain system of claim 9, wherein the electronic control system is configured to set the fuel composition parameter as one of the first biodiesel content value, the second biodiesel content value, the second biodiesel content value, and an average of two or more of the first biodiesel content value, the second biodiesel content value, and the third biodiesel content value.

11. A method of controlling a powertrain system, the method comprising:
receiving with an electronic control system an output of a fuel sensor system, the output being indicative of a plurality of characteristics of fuel in a fuel supply system adapted to supply fuel to an engine, the plurality of characteristics including information indicative of a fuel temperature and one or more of a fuel density, a fuel viscosity, and a fuel dielectric,
determining a fuel composition parameter by processing the output with the electronic control system, and
controlling with the electronic control system one or more operations of the powertrain system, the controlling utilizing the fuel composition parameter.

12. The method of claim 11, wherein the plurality of characteristics include information indicative at least two of the fuel density, the fuel viscosity, and the fuel dielectric, and the electronic control system is configured to check for an error condition by evaluating at least said at least two of the fuel density, the fuel viscosity, and the fuel dielectric.

13. The method of claim 12, comprising:
determining with the electronic control system a first biodiesel content value using the fuel temperature and a first one of the at least two of the fuel density, the fuel viscosity, and the fuel dielectric, and
determining with the electronic control system a second biodiesel content value using the fuel temperature and a second one of the at least two of the fuel density, the fuel viscosity, and the fuel dielectric.

14. The method of claim 13, comprising evaluating with the electronic control system the first biodiesel content value and the second biodiesel content value to check for the error condition.

15. The method of claim 13, comprising determining the fuel composition parameter using the first biodiesel content value and the second biodiesel content value.

16. The method of claim 13, comprising setting the fuel composition parameter as one of the first biodiesel content value, the second biodiesel content value, and an average of the first biodiesel content value, and the second biodiesel content value.

17. The method of claim 11, wherein the plurality of characteristics include information indicative all of the fuel density, the fuel viscosity, and the fuel dielectric, and the electronic control system is configured to check for an error condition by comparing all of the fuel density, the fuel viscosity, and the fuel dielectric.

18. The method of claim 17, wherein comprising determining a first biodiesel content value using the fuel temperature and the fuel density, a second biodiesel content value using the fuel temperature and the fuel viscosity, and a third biodiesel content value using the fuel temperature and the fuel dielectric.

19. The method of claim 18, comprising utilizing the first biodiesel content value, the second biodiesel content value, and the third biodiesel content value to determine to determine the fuel composition parameter.

20. The method of claim 19, comprising setting the fuel composition parameter as one of the first biodiesel content value, the second biodiesel content value, the second biodiesel content value, and an average of two or more of the first biodiesel content value, the second biodiesel content value, and the third biodiesel content value.
